(19) United States
(12) Patent Application Publication
(10) Pub. No.: US 2024/0122637 A1
(43) Pub. Date: Apr. 18, 2024

(54) Surgical Method for Reducing Insulin Resistance

(71) Applicant: B2M MEDICAL, INC., Irvine, CA (US)

(72) Inventors: Rafi MAZOR, San Diego, CA (US); Alexei V. Babkin, Dana Point, CA (US)

(21) Appl. No.: 18/485,717

(22) Filed: Oct. 12, 2023

Related U.S. Application Data
(60) Provisional application No. 63/443,696, filed on Feb. 6, 2023.
(60) Provisional application No. 63/416,606, filed on Oct. 17, 2022.

(51) Int. Cl. A61B 18/02; A61B 5/00; A61B 5/055; A61B 5/145

(52) U.S. Cl. CPC A61B 18/02; A61B 5/055; A61B 5/14532; A61B 5/4872; A61B 2018/0212; A61B 2018/0231

(57) ABSTRACT

A surgical method for reducing insulin resistance comprises cryolipolysis of mesenteric fat in a controlled manner to avoid tissue ablative temperatures. Cryolipolysis is carried out in stages based on evaluating reduction in volumes of mesenteric fat and fasting glucose levels.

### CROSS-REFERENCES TO RELATED APPLICATIONS

This claims priority to provisional application No. 63/416,606, filed Oct. 17, 2022, entitled “Surgical Method and Apparatus for Reducing Insulin Resistance” and to provisional application No. 63/443,696, filed Feb. 6, 2023, entitled “Surgical Method and Apparatus for Reducing Insulin Resistance” each of which is incorporated herein by reference in its entirety for all purposes.

### GOVERNMENT SPONSORSHIP

This invention was made with government support under Award Number R43DK133015 awarded by National Institute of Diabetes and Digestive and Kidney Diseases of the National Institutes of Health. The government has certain rights in the invention

### FIELD OF THE INVENTION

The inventions described herein relate to surgical methods for treating insulin resistance.

### BACKGROUND OF THE INVENTION

The prevalence of type II diabetes mellitus (T2DM), insulin resistance, and obesity is continuously rising and poses a major risk for morbidity and mortality from cardiovascular and other complications. Studies in animal models and humans have shown a link between visceral (but not subcutaneous) fat and insulin resistance and related metabolic abnormalities. Among the fat depots, the mesentery stores most of the intra-abdominal fat, and has emerged as a key contributor to insulin resistance and diabetes via recruitment of inflammatory cells and the secretion of adipokines, cytokines, free fatty acids, and other diabetogenic factors. Moreover, increase in mesenteric fat thickness is closely linked to increased risk for developing the metabolic syndrome (MetS), defined as the presence of at least three of the following features: central obesity, high blood pressure, hyperglycemia, low high-density lipoprotein cholesterol (HDL-c), and elevated serum triglycerides. The direct drainage of the mesentery to the liver contributes to the development of fatty liver and liver-specific insulin resistance which in many cases antecede systemic insulin resistance and diabetes.

Mesenteric fat, however, is generally unresectable surgically due to its vital neurovascular supply to the intestines. It cannot be surgically removed without compromising the vitality of the intestines.

Currently, there are no clinically available treatments to reduce the deleterious mesenteric fat mass, and alternate strategies which include dietary and lifestyle approaches are clearly not sustainable in most cases.

Accordingly, there is still a need for an improved method and apparatus that overcomes the above-mentioned shortcomings.

### SUMMARY OF THE INVENTION

An embodiment of the invention is a surgical method of treating insulin resistance in a patient. The method comprises obtaining a current measurement of at least one of a plurality of insulin resistance indicators; accessing the mesenteric fat; and performing a first stage of cryolipolysis on the mesenteric fat with a cryodevice sufficient to cause the at least one of the plurality of insulin resistance indicators to reach a threshold amount.

In embodiments, the insulin resistance indicators are at least one of the following: fasting glucose, mesenteric fat volume, hemoglobin A1C, and fasting insulin.

In embodiments, the threshold amount is present at least 3, and more preferably, at least 6 months from the first stage of cryolipolysis.

In embodiments, the at least one of the plurality of insulin resistance indicators is volume of mesenteric fat, and is measured using CT or MRI.

In embodiments, a second stage of cryolipolysis is performed if the difference between the initial volume of mesenteric fat and remeasured volume of mesenteric fat is less than a reduced fat threshold amount. In embodiments, the reduced fat threshold amount is 30%.

In embodiments, a second stage of cryolipolysis is performed if a difference between an initial fasting glucose level and a remeasured fasting glucose level is less than a fasting glucose threshold amount. In embodiments, the fasting glucose threshold amount is 20%.

In embodiments, the first stage of cryolipolysis comprises visually observing the tissue surface for changes in appearance, and terminating the first stage based on the observed changes in tissue appearance. Optionally, the visually observing comprises observing for ridges or folds and color changes.

In embodiments, the method comprises the step of creating an incision in the abdomen to provide open-surgery type access to the mesenteric fat.

In embodiments, the method comprises performing a second stage of cryolipolysis.

In embodiments of the invention, a method of treating insulin resistance in an animal comprises the step of cryolipolysis of mesenteric fat.

In embodiments of the invention, a method of treating metabolic syndrome in an animal comprises the step of cryolipolysis of mesenteric fat.

In embodiments of the invention, a method of reducing blood pressure in an animal comprises the step of cryolipolysis of mesenteric fat.

In embodiments, a surgical method of reducing systolic blood pressure comprises: initially measuring at least one insulin resistance indicator in the patient; accessing mesenteric fat of the patient; and carrying out a first stage of cryolipolysis of the mesenteric fat using a cryodevice such that the at least one insulin resistance indicator is reduced by a threshold amount, thereby reducing SBP.

In embodiments, the threshold amount is measured at least 3 months after the first stage.

In embodiments, the at least one insulin resistance indicator is selected from the group consisting of fasting glucose, mesenteric fat volume, and fasting insulin.

In embodiments, the at least one insulin resistance indicator is mesenteric fat volume, and the mesenteric fat volume threshold amount is 30%.

In embodiments, the at least one insulin resistance indicator also comprises fasting glucose, and optionally hemoglobin A1C.

In embodiments, the fasting glucose threshold amount is 20%.

In embodiments of the invention, a method of improving glycemic control in an animal comprises the step of cryolipolysis of mesenteric fat.

In embodiments of the invention, a method of reducing the volume of mesenteric fat in an animal comprises the step of cryolipolysis of the mesenteric fat.

In embodiments of the invention, the step of cryolipolysis reduces the volume of mesenteric fat by at least about 30 percent, as measured at least 3 months after the step of cryolipolysis was performed.

In embodiments of the invention, the step of cryolipolysis is performed using a handheld probe operable to cool the mesenteric fat, and optionally, to cool the mesenteric fat by releasing a gas through a nozzle aimed at the mesenteric fat.

In embodiments, the nozzle has a circular shower head-like shape.

In embodiments of the invention, the step of cryolipolysis is performed via a laparoscopic or open surgical approach.

In embodiments of the invention, the step of cryolipolysis is performed such that tissue ablative temperatures are avoided.

In embodiments of the invention, the method comprises the step of exposing a portion of the mesentery prior to the step of cryolipolysis.

In embodiments of the invention, the step of cryolipolysis is performed without damaging the neurovascular bundles to the organs.

The description, objects and advantages of the present invention will become apparent from the detailed description to follow, together with the accompanying drawings.

### BRIEF DESCRIPTION OF THE DRAWINGS

The description, objects and advantages of embodiments of the present invention will become apparent from the detailed description to follow, together with the accompanying drawings.

FIGS. 1A-1C depict cryolipolysis procedures on exposed tissue of an Ossabaw pig using, respectively, a 3″ diameter circular-big probe design, 2.5″ square/rectangular probe design, and a 1.5″ diameter circular-small probe design;

FIG. 1D depicts treated and untreated tissue using the probe shown in FIG. 1A;

FIG. 2A depicts CT images showing segmentation of the mesenteric fat in a cryolipolysis treated pig before treatment (left) and the same pig at 6 months follow up (right).

FIG. 2B is a chart showing volume of mesenteric fat in Sham and cryolipolysis-treated pigs before treatment and at 3 and 6 months follow up.

FIG. 2C depicts mesenteric fat of sham (left) and cryolipolysis-treated (right) at 6 months follow up, showing marked reduction in the thickness while no apparent damage to the microcirculation or the intestine.

FIGS. 3A, 3B are charts showing changes in total body weight and Systolic Blood Pressure (SBP), respectively, in Sham and cryolipolysis treated before treatment (T=0) and at 3 and 6 months follow up.

FIGS. 4A-4D are charts, respectively, for fasting plasma glucose, insulin, HOMA-IR, and QUICKI before treatment and at 3 and 6 months follow up.

FIG. 5A is a chart showing plasma glucose levels measured before glucose administration and at 5, 10, 20, 30, 40, 50 and 60 min thereafter for both groups before cryolipolysis and at 3 and 6 months follow up.

FIG. 5B is chart showing the area under the curve shown in FIG. 5A for each group at the respective follow up period.

FIG. 5C is a chart showing plasma insulin levels measured before glucose administration and at 10, 20, 40, and 60 min thereafter for both groups before cryolipolysis and at 3 and 6 months follow up.

FIG. 5D is chart showing the area under the curve shown in FIG. 5C for each group at the respective follow up period

FIGS. 6A-6B depict an intestine at 6 months follow up, showing minor local adhesions on the intestine serosa (indicated by the circle) in both cryolipolysis treated and sham control, respectively.

FIG. 6C depicts a histology sample of mesenteric fat and the small intestine cross section from a cryolipolysis treated pig at 6 months follow up. Bar=5 mm.

### DETAILED DESCRIPTION OF THE INVENTION

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described. It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

All existing subject matter mentioned herein (e.g., publications, patents, patent applications and hardware) is incorporated by reference herein in its entirety except insofar as the subject matter may conflict with that of the present invention (in which case what is present herein shall prevail). The following are incorporated by referenced in their entirety for all purposes: Methods and systems for cooling visceral fat while leaving surrounding tissue unharmed are disclosed in our prior International Patent Publication WO 2020/061202 (published Mar. 26, 2020); International Patent Publication WO 2021102301 (published May 27, 2020); and International Application No. PCT/US22/82674 (filed Dec. 31, 2022) entitled “METHOD AND SYSTEM FOR MINIMALLY INVASIVE REMOVAL OF MESENTERIC FAT”.

Described herein are various methods and apparatuses for treating insulin resistance in animals. By animals, it is meant to include, without limitation, humans.

In preferred embodiments of the invention, a novel cryolipolysis method and cryodevice comprises obtaining a current measurement of at least one of a plurality of insulin resistance indicators; accessing the mesenteric fat; and performing a first stage of cryolipolysis on the mesenteric fat with the cryodevice sufficient to cause the at least one of the plurality of insulin resistance indicators to reach a threshold amount. In embodiments, the threshold amount is measured at least 3 months, optionally 6 months, after the first stage.

Examples of insulin resistance indicators include, without limitation, fasting glucose, mesenteric fat volume, fasting insulin, and hemoglobin A1C.

The step of obtaining measurements may be performed using various techniques depending on the insulin resistance indicator being measured. For example, measurement of mesenteric fat volume may be obtained using CT, MRI scans. Measurements of the fasting blood glucose, A1C, and insulin levels may be obtained from conventional lab panels or assay blood sample tests.

The step of accessing the mesenteric fat may be performed using a laparoscopic approach, or open surgical approach. Either approach can expose the mesentery for cryolipolysis.

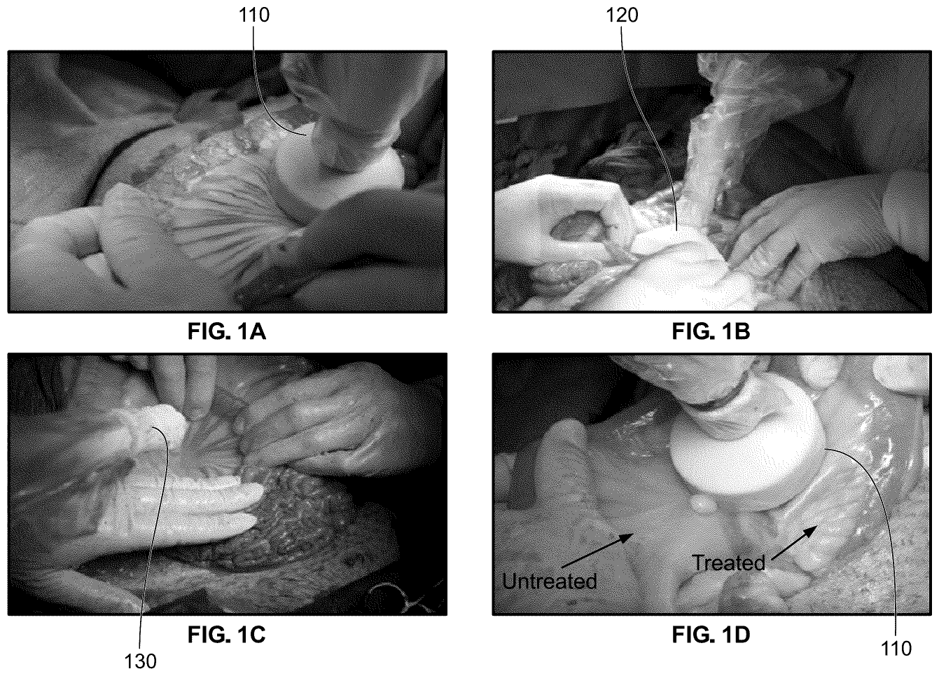

Cryolipolysis is performed by a cryodevice or probe and in preferred embodiments, without ablation of the mesenteric fat. Preferred embodiments of the invention are non-ablative in nature. The configuration of the cryodevice (e.g., cryodevice 110, 120, 130 of FIGS. 1A-1C) may vary. In embodiments of the invention, it is a handheld probe operable to cool the mesenteric fat, and optionally, to cool the mesenteric fat by releasing a gas through a nozzle aimed at the mesenteric fat. In embodiments, the nozzle has a circular shower head-like shape.

Regardless of the specific configuration of the cryodevice, it is placed in contact or adjacent to the mesenteric fat and activated.

The tissue being treated is observed for visual indicators for doneness or completeness. Examples of visual indicators include folds, ridges, color changes. In an embodiment, the physician treats the target tissue until the color changes from pink to white and then moves the device to another location. For example, with reference to FIG. 1D, cryodevice 110 is shown applying energy to the “treated” tissue which has become ridged and white versus the “untreated” relatively smooth dark colored tissue.

In preferred embodiments of the invention, cryolipolysis is performed sufficient to achieve the threshold amount. The threshold amount or value can vary depending on the type of insulin resistance indicator. For example, if the insulin resistance indicator is mesenteric fat volume, a preferred mesenteric fat volume threshold amount is at least 30%. That is, the volume of mesenteric fat is reduced by at least 30% in embodiments of the invention after the first stage of cryolipolysis. However, the invention is not so limited. For other types of insulin resistance indicators, the threshold amount can be at least 10% or at least 20%.

In embodiments, additional stages of cryolipolysis are performed if the insulin resistance indicator is not below (or within) the threshold value. For example, if the indicator is hemoglobin A1C, an additional stage of cryolipolysis is performed if the measured A1C is not less than 6.4% and in some embodiments, is not less than 5.7%.

In some embodiments of the invention, a plurality of insulin resistance indicators are evaluated and averaged, and the average of the group of insulin resistance indicators is compared before and after the first stage of cryolipolysis. In embodiments, the method comprises a set of computer-implemented logic rules for weighting volumetric fat reduction greater than other indicators. In some embodiments, a computer system is programmed and operable to compute a score for a plurality of insulin resistance indicators for the effectiveness of the cryolipolysis and if the score is less than a goal value, a second stage of lipolysis is performed.

In view of the above, and disclosure herein, the invention is intended to include many different embodiments in which the following is an exemplary embodiment for performing a procedure on an animal for treating insulin resistance and high blood pressure.

### Example 1

The following is an example for treating swine in accordance with embodiments of the invention.

MetS and insulin resistance were induced in 8 Ossabaw miniature swine (5 Males/3 Females) with a daily atherogenic diet (1000 g), high in fat, cholesterol and fructose for 9 months starting at 9 months of age. The diet provided 16.3% kcal from protein, 40.8% kcal from total carbohydrates (for which 19% kcal was fructose), and 42.9% kcal from fat. Fat calories were derived from a mixture of lard, hydrogenated soybean oil, and hydrogenated coconut oil. It was supplemented with 2.0% cholesterol and 0.7% sodium cholate by weight (KT324, Purina Test Diet, Richmond, IN). All animals had free access to water.

Study design. After 9 months of a high fat diet (HFD), abdominal computed tomography (CT, described below) was performed to quantify the mesenteric fat. Blood was drawn and intravenous glucose tolerance test (IVGTT) was performed to establish baseline insulin resistance. Mesenteric fat cryolipolysis (MFC), described below was performed on 5 pigs while 3 pigs underwent laparotomy and manipulation of their mesentery without cooling and served as sham controls. All surgical procedures and imaging as described below were identical between the groups, however, the cryolipolysis device was not activated in the sham group. Pigs were allowed to recover and monitored up to 6 months. CT and glucose monitoring were conducted at 3 and 6 months follow up. At 6 months, pigs were euthanized, and a necropsy was performed.

Cryolipolysis. The pigs were randomly divided into sham (2M, 1F) and treated (3M/2F) groups. After an overnight fast, swine received a 2.2 mg/kg dose of xylazine (Webster Veterinary, Devens, MA) and 5.5 mg/kg dose of telazol (Fort Dodge Animal Health, Fort Dodge, IA) intramuscular injection to induce anesthesia. Swine were intubated and anesthesia was maintained with 2-4% isoflurane in 100% O₂ as a carrier gas. The isoflurane level was adjusted to maintain anesthesia with stable hemodynamics. Heart rate, blood pressure, respiratory rate, and electrocardiographic data were continuously monitored throughout the procedure. A laparotomy was performed to expose the mesenteric fat of the small intestine. The average thickness of the mesenteric fat in these pigs was ˜5 mm, which is comparable to that of humans.

Cryolipolysis device. A cryolipolysis device as described herein was provided. The device used compressed nitrogen gas cooled to −190° C. by liquid nitrogen and then heated to the treatment temperature by heating elements located in the device probe before being released through the probe nozzle. Based on preliminary bench testing and measurements of temperature penetration to fat tissue, we set the treatment parameters to cool the mesenteric fat to ≤10° C. at a depth of 5 mm. We identified the terminal ileum as the starting point for cryo-applications and treated the mesenteric fat in a retrograde direction throughout the small intestine until we reached the duodenum. Since the anatomy of the pig's spiral colon does not allow access to its mesentery, we avoided treating the large intestine.

Computed tomography. CT scans were conducted before cryolipolysis and at 3 and 6 months follow up to quantify changes in mesenteric fat, defined as fat engulfing the intestine excluding air. The pigs were scanned using a Philips Brilliance 64 detector scanner (Philips Healthcare, Andover, MA, USA and Best, NL) while sedated using 4% Isoflurane (Webster Veterinary, Devens, MA) supplied by mask. Helical scans were obtained craniocaudal from the sternal notch to the pubic symphysis, 20 mm thick and taken every 0.5 cm. Images were analyzed using Mimics Medical 24.0 (Materialize NV, Belgium), with Hounsfield units (HU) range of −150 to −20. The mesenteric fat was segmented, and its volume (L) was calculated from the 3D representations.

Intravenous glucose tolerance test (IVGTT). After an overnight fast, pigs were anesthetized with isoflurane and the right jugular vein was catheterized percutaneously. They were allowed to recover from anesthesia for at least 2 h before the IVGTT started, to avoid isoflurane induced decrease in insulin action. The animals were placed in a low-stress restraint sling and blood samples were obtained at baseline (−5 and 0 min), followed by an intravenous bolus of 1 g glucose/kg body weight, and further samples were obtained at 5, 10, 20, 30, 40, 50, and 60 min after injection. Blood glucose was measured on a YSI 2300 Stat Plus analyzer (YSI, Yellow springs, OH). Plasma insulin levels were obtained by Elisa assays (Abcam, MA, USA) at 0, 10, 20, 40 and 60 min after injection of glucose. To assess insulin sensitivity, the products of plasma glucose and insulin levels were used to calculate a modified homeostatic model assessment of insulin resistance (HOMA-IR), and Quantitative Insulin Sensitivity Check Index (QUICKI).

Blood pressure during IVGTT was monitored using the cuff method as previously described, and values were computed from at least 6 different measurements over time.

Statistical Analysis: Data presented as mean±SEM unless otherwise indicated. The area under the curve (AUC) for glucose and insulin was calculated using the trapezoid method. 2-way anova for repeated measurements followed by post-hoc Bonferroni test for comparisons between groups was used to measure significance within groups. Student T test was used to compare values between groups at different time points. p<0.05 is considered as statistically significant.

Results

Cryolipolysis. All cryolipolysis and sham procedures were completed without any adverse events. Overall, we delivered ˜40-60 applications per pig and the procedure time was ˜90 minutes. No stigmata of intrabdominal bleeding were recorded, and all the pigs had regular bowel movements within 24 h after the procedure. Three different designs of our prototype cryolipolysis probe allowed access to most of the visible mesenteric fat along the entire length of the small intestine (FIG. 1A-C). Following cryo-application, the treated area underwent a transient pale discoloration with a firmer consistency compared to untreated tissue, until baseline body temperature was restored in that location (FIG. 1D). We used this change in appearance to identify treated vs. untreated areas of the mesentery as we progressed to treat majority of the mesenteric surface.

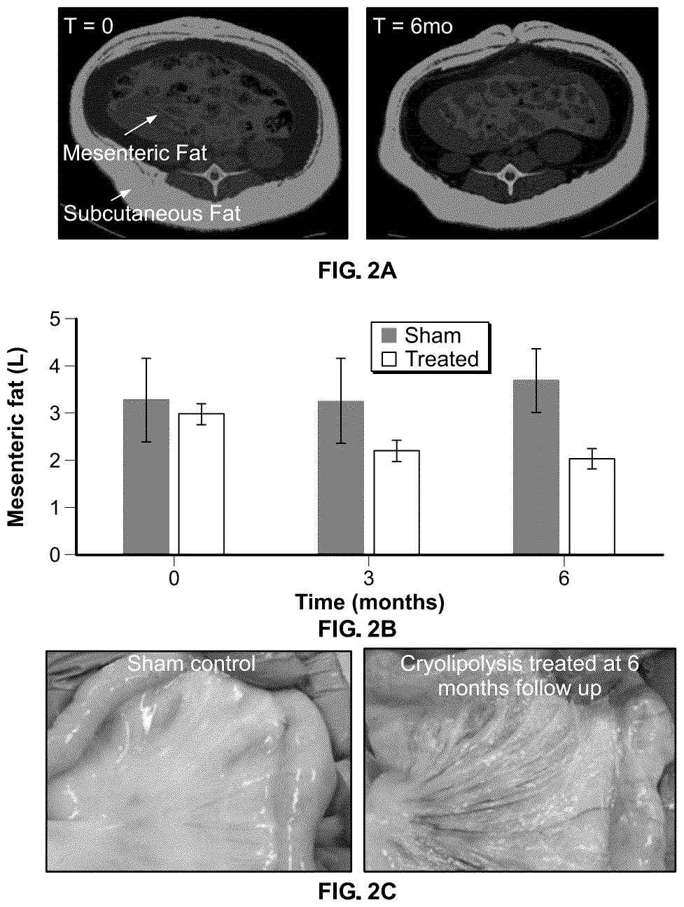

Mesenteric fat. With reference to FIGS. 2A, 2B, before cryolipolysis (T=0), the mean volume of the mesenteric fat in the treated and sham groups was (2.98±0.23 L vs 3.28±0.87 L, respectively; p=ns). At 3 months post-cryolipolysis, mesenteric fat volume was reduced by ˜30% in the treated group, while it did not change significantly in the sham controls (2.22±0.19 L vs. 3.26±0.9 L, respectively; p=0.009). The reduction of mesenteric fat volume was sustained at 6 months (p=0.003) while the sham controls showed a 20% increase (2.03±0.22 L vs 3.7±0.65 L, respectively,). A representative segment of treated vs. untreated mesenteric fat taken at necropsy at 6 months shows a marked reduction in the thickness of the mesenteric fat following cryolipolysis (FIG. 2C) without thrombotic or atrophic mesenteric vasculature.

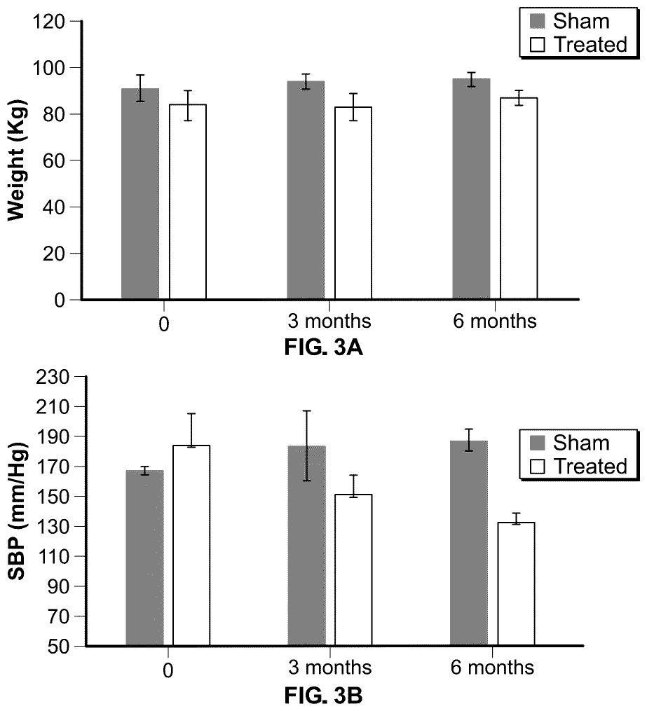

Body weight. Before the initiation of 9 months HFD, body weight was not different between the groups (32±4 kg vs. 30±3 kg, sham vs. treated, p=n.s). MFC had no significant effect on total body weight, which increased by ˜4 kg in both groups at 6 months follow-up (sham: 91.2±5.6 kg at t=0 to 95.1±3 kg at t=6 months; treated: 84±6.5 kg at t=0 to 87.1±3.5 kg at t=6 months; FIG. 3A).

Blood pressure. Before the initiation of 9 months HFD, blood pressure was not different between the groups (109±5 mmHg vs. 108±11 mmHg, sham vs. treated, p=n.s). Before cryolipolysis (T=0), both sham and treatment groups were hypertensive with systolic blood pressure (SBP) of 167±4 mmHg and 183±22 mmHg respectively. Mesenteric fat cryolipolysis reduced SBP in the treated group by 17% to 150±14 mmHg at 3 months follow up (p=0.02), and these levels decreased further by 28% at 6 months follow up to 132±6 mmHg (p=0.007). There was no significant change in SBP values in the sham control which remained elevated at 6 months follow up (183±23 mmHg at 3 months and 187±7 mmHg at 6 months; p=ns; FIG. 3B).

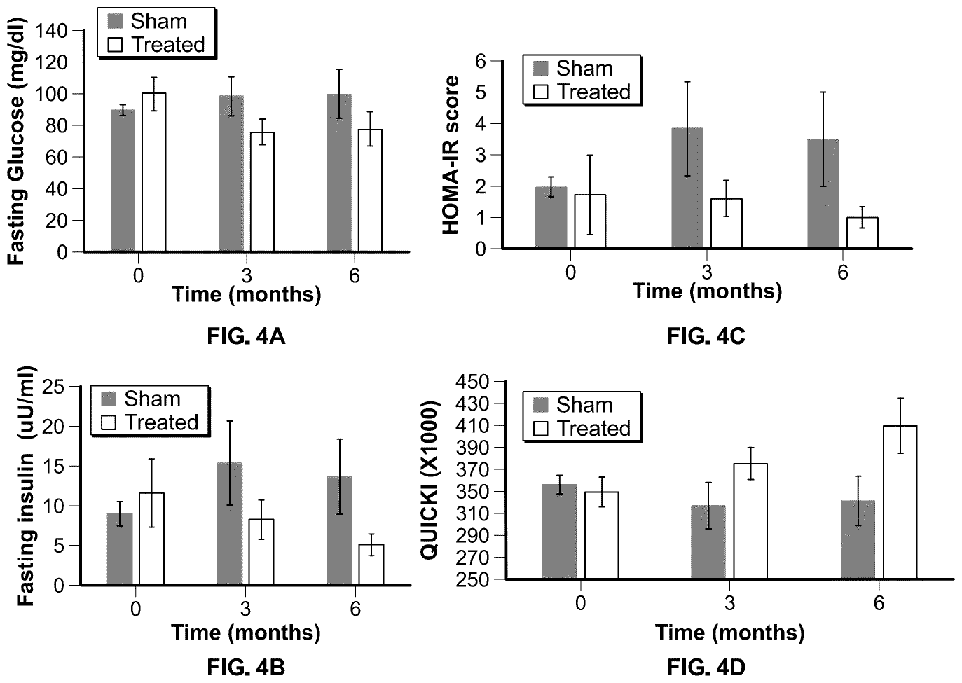

Fasting Glucose and Insulin levels. Before the initiation of 9 months HFD, fasting blood glucose was not different between the groups (64±7 mg/dL vs. 68±7 mg/dL, sham vs. treated, p=n.$). Before cryolipolysis (T=0), the mean values of fasting glucose and fasting insulin in the treated and sham groups were 89±11 and 99±4 mg/dL for glucose; 11.5±4.3 and 9±1.5 uU/ml for insulin, respectively; p=ns. At 3 months post cryolipolysis, these levels were reduced in the treated group to 75±8 mg/dL for glucose and 8.2±2.4 uU/ml for insulin (p<0.05). In the sham group, mean fasting glucose and insulin concentrations tended to increase at 3 and 6 months compared with values at baseline (T=0), but the differences were not statistically significant. In contrast, fasting plasma glucose and insulin concentrations were lower at 6 months compared with baseline in the MFC group (FIG. 4A, 4B).

HOMA-IR and QUICKI scores. Before cryolipolysis (T=0), the mean values of HOMA-IR and QUICKI were not significantly different between the sham and treated groups (HOMA-IR values: 1.98±0.32 and 1.72±0.2, p=ns; QUICKI(×1000): 346±9.36 and 339±14.53, p=ns, respectively). At 3 months post cryolipolysis, HOMA-IR values reduced to 1.61±0.57 in the treated group while increased to 3.83±1.51 in the sham control (p<0.05). At 6 months, HOMA-IR further reduced in the treated group to 1.0±0.34 (p<0.05), while it remained elevated at 3.5±1.51 in the sham control. QUICKI score increased at 3 months in the cryolipolysis treated group while it remained reduced in the sham control [368±15.65 vs. 325±23.23 respectively]. At 6 months follow up, QUICKI further increased in the treated group to 406±27.42 (p=0.01) while it was unchanged in the sham control (330±22.88; FIG. 4C, 4D).

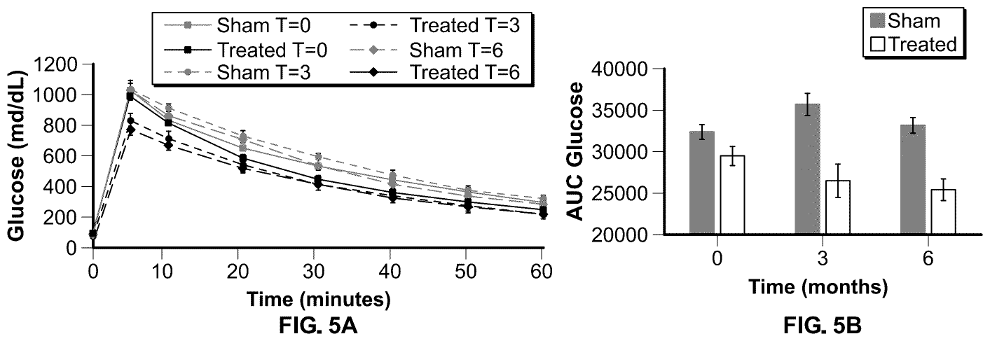

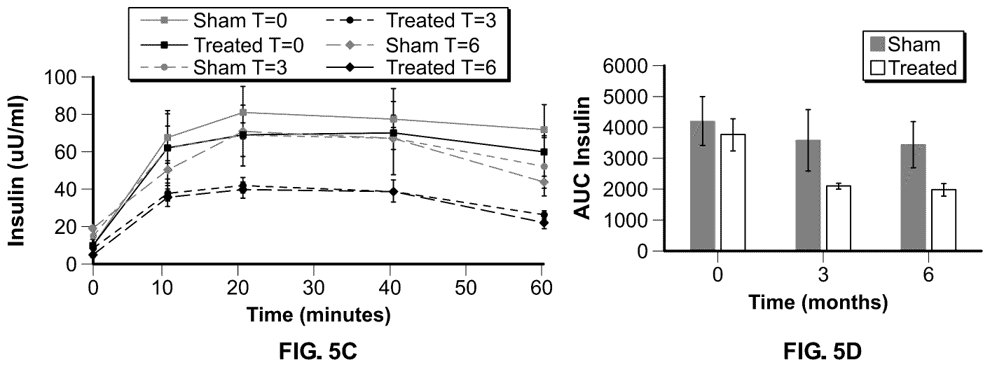

Glucose and Insulin levels during IVGTT. Glucose: Before cryolipolysis (T=0), the peak value as well as the area under the curve (AUC) for glucose were not significantly different between the sham and treated groups (AUC values: 32402±908.23 vs. 29169±1162 respectively; p=ns). At 3 months post mesenteric fat cryolipolysis, both the peak values and the AUC for glucose were reduced compared to the sham control (AUC values: 26527±2054 vs. 35764±1382, p=0.009). These reduced values were also sustained at 6 months follow up (AUC values: 25452±1890 vs 33275±1869; treated vs. sham, respectively; p=0.01) (FIG. 5A, 5B). Insulin: Before cryolipolysis (T=0), the peak value as well as the area under the curve (AUC) for insulin were not significantly different between the sham and treated groups (AUC values: 4218±787 vs. 3772±521 respectively; p=ns). Three months following mesenteric fat cryolipolysis, both the peak values and the AUC for insulin were reduced compared to the sham control (AUC values: 2096±101 vs. 3598±989). These reduced values were sustained at 6 months follow up (AUC values:1989±200 vs. 3456±741; p=0.02, FIG. 5C, 5D).

Safety and Pathology. There were no adverse events at all stages of the study in response to MFC. All pigs recovered from the procedure with no difference in food/water consumption or bowel movements compared to the sham controls. At necropsy, a subjective, relative score assessing the degree of fibrous adhesions in the mesentery was performed by a pathologist. The score ranged from 0 (no adhesions) to 10 (widespread) and all the pigs were scored between 0 to 3.

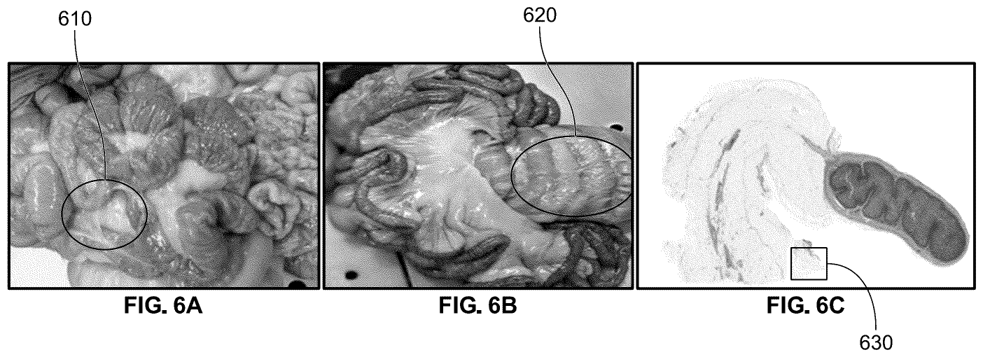

With reference to FIGS. 6A-6B, gross pathology showed minor local adhesions (in both treated and sham groups 610, 620, respectively) along the intestine with no apparent physiological relevance. Examination of internal organs such as adjacent bowel, heart, liver, kidney, and spleen showed no signs of damage, and they were comparable to sham controls.

FIG. 6C depicts an H&E histology sample of mesenteric fat and the small intestine cross section 630 from a cryolipolysis treated pig at 6 months follow up. The mesentery shows minimal areas of benign fibrous adhesions on the surface (boxed area). Bar=5 mm.

### DISCUSSION

In the above example, we demonstrated the safety and therapeutic metabolic efficacy of mesenteric fat cryolipolysis on glycemic control and indices of insulin resistance in the Ossabaw swine model of HFD induced MetS. We found MFC can decrease the volume of the mesenteric fat by ˜30% at 3 months follow up, which was sustained at 6 months post procedure and was associated with a decrease in BP, fasting blood glucose, glucose AUC after glucose infusion and insulin resistance. Moreover, the cryolipolysis procedures produced no early or late complications with all animals surviving to the termination of the study.

Without intending to being bound to theory, adipocytes, due to their lipid content, are sensitive to low temperatures compared to other cell types, and induction of cell death can be detected at temperatures as high as +10° C. This relatively high temperature avoids tissue ablation or damage to other cells or adjacent vital structures. Thus, targeting fat cells for destruction using cold temperatures (cryolipolysis) is an attractive approach to induce mesenteric fat loss.

Following the cryolipolysis we performed as described above, we found on average a ˜30% decrease in mesenteric fat volume measured at 3- and 6-months post procedure. This amount of fat loss was sufficient to demonstrate a durable reduction in glucose dysregulation indices, namely fasting glucose, and insulin levels, as well as glucose and insulin concentrations during the intravenous glucose tolerance test, without a decrease in body weight.

An unexpected result was the reduction in blood pressure following the procedure. This reduction was not associated with any appreciable reduction in total body weight and, to our knowledge, this effect of mesenteric fat reduction on hypertension has not been reported.

We also found that mesenteric fat cryolipolysis did not result in weight loss, as all the animals modestly gained weight by the 6 months follow up. One possible explanation may be our pigs consumed all their daily feed within 24 h of their procedures. Thus, our results show that improvements in glycemic control after mesenteric fat cryolipolysis are achieved independently from weight reduction when applied to obese pigs. We do however expect that in human subjects with visceral obesity, some weight reduction will occur in alignment with the improvement in the overall metabolic status of the patient. The above example is evidence that MFC is a safe and effective method to remove mesenteric fat, and that this reduction will promote sustained improvements in glycemic control.

We also note that although we found mesenteric fat reduction to be durable at 6 months follow up, both treated and sham groups gained weight. Since the diet did not change during the follow up period, this result could be explained by fat redistribution to other fat depots such as subcutaneous fat. Thus, we used our CT scans to measure the volume of the subcutaneous fat around the abdomen area and found no differences between the time points. Other parts which are known in pigs to accumulate subcutaneous fat like the neck and limbs were not scanned. Thus, and without intending to be bound by theory, the reduction in mesenteric fat in pigs promoted fat redistribution to other parts, mainly subcutaneous depots.

The above example showed in the Ossabaw swine, which represents a highly translatable model that develops each of the core parameters of the MetS with many of the associated secondary comorbidities, that delivering non-ablative cold temperatures to the mesenteric surface promotes fat loss concomitant with reduction in insulin resistance and MetS indices. We additionally showed that in pigs, the procedure is safe and durable for at least 6 months.

While the preferred embodiments of the devices and methods have been described in reference to the environment in which they were developed, they are merely illustrative of the principles of the inventions. For example, although a laparotomy approach is described above, the invention can include other approaches such as laparoscopic approach except where limited by any recited claims. Additionally, although an open-loop cryo-system was described above, the invention can include other configurations such as a closed-loop cryo-system in which cooling is performed based on thermal conduction through a thermally conductive wall or barrier. Additionally, the elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

1. A surgical method of treating insulin resistance in a patient comprising:
obtaining a current measurement of at least one of a plurality of insulin resistance indicators;
accessing mesenteric fat of the patient; and
performing a first stage of cryolipolysis with a cryodevice on the mesenteric fat sufficient to cause the at least one of the plurality of insulin resistance indicators to reach a threshold amount, thereby reducing insulin resistance.

2. The surgical method of claim 1, further comprising evaluating for whether the at least one of the plurality of insulin resistance indicators reaches the threshold amount is performed at least 6 months from the first stage of cryolipolysis.

3. The surgical method of claim 1, wherein the at least one of the plurality of insulin resistance indicators is volume of mesenteric fat, and is measured using CT or MRI.

4. The surgical method of claim 3, wherein a second stage of cryolipolysis is performed if the difference between the initial volume of mesenteric fat and remeasured volume of mesenteric fat is less than a reduced fat threshold amount.

5. The surgical method of claim 4, wherein the reduced fat threshold amount is 30%

6. The surgical method of claim 1, wherein a second stage of cryolipolysis is performed if a difference between an initial fasting glucose level and a remeasured fasting glucose level is less than a fasting glucose threshold amount.

7. The surgical method of claim 6, wherein the fasting glucose threshold amount is 20%.

8. The surgical method of claim 1, wherein the first stage of cryolipolysis comprises visually observing the tissue surface for changes in appearance, and terminating the first stage based on the observed changes in tissue appearance.

9. The surgical method of claim 8, wherein the visually observing comprises observing for ridges or folds and color changes.

10. The surgical method of claim 1, wherein the cryodevice is a handheld probe.

11. The surgical method of claim 10, wherein the cryodevice is operable to cool the mesenteric fat by releasing a gas through a nozzle aimed at the mesenteric fat.

12. The surgical method of claim 11, wherein the nozzle has a circular shower head-like shape.

13. The surgical method of claim 1, further comprising the step of creating an incision in the abdomen to provide open-surgery type access to the mesenteric fat.

14. The surgical method of claim 1, wherein the cryolipolysis is performed via a laparoscopic approach.

15. The surgical method of claim 1, wherein the cryolipolysis is performed such that tissue ablative temperatures are avoided.

16. The surgical method of claim 1, further comprising performing the second stage of cryolipolysis.

17. The surgical method of claim 2, wherein the evaluation is performed by a computer processor.

18. A surgical method of reducing systolic blood pressure comprising:
initially measuring at least one insulin resistance indicator in the patient;
accessing mesenteric fat of the patient; and
carrying out a first stage of cryolipolysis of the mesenteric fat using a cryodevice such that the at least one insulin resistance indicator is reduced by a threshold amount, thereby reducing SBP.

19. The surgical method of claim 18, wherein the threshold amount is measured at least 3 months after the first stage.

20. The surgical method of claim 19, wherein the at least one insulin resistance indicator is selected from the group consisting of fasting glucose, mesenteric fat volume, and fasting insulin.
